(19) United States
(12) Patent Application Publication
(10) Pub. No.: US 2025/0319234 A1
(43) Pub. Date: Oct. 16, 2025

(54) SMART CUSTOMIZABLE THERAPEUTIC MECHANISMS AND SYSTEMS FOR TISSUE REGENERATION AND GROWTH

(71) Applicant: Smartweave, Inc, Boston, MA (US)

(72) Inventors: Rajeev Malhotra, Newton, MA (US); Jorge Alexis Mojica Santiago, Houston, TX (US)

(73) Assignee: Smartweave, Inc, Boston, MA (US)

(21) Appl. No.: 19/064,369

(22) Filed: Feb. 26, 2025

Related U.S. Application Data
(60) Provisional application No. 63/557,640, filed on Feb. 26, 2024.

(51) Int. Cl. A61L 27/54; A61L 27/56; A61L 27/58

(52) U.S. Cl. CPC A61L 27/54; A61L 27/56; A61L 27/58; A61L 2430/02; A61L 2430/06; A61L 2430/10; A61L 2430/12; A61L 2430/32; A61L 2430/34

(57) ABSTRACT

An embodiment herein provides a therapeutic mechanism for tissue regeneration and therapeutic monitoring. The therapeutic mechanism may include a biocompatible ink configured to transition from an initial state to a structured form in response to interaction with predefined physiological stimuli post injection into a target recipient. The therapeutic mechanism may include one or more therapeutic agents and a bio-sensing matter that may be configured to detect at least one biological parameter of the target recipient post transition of the biocompatible ink into the structured form.

### CLAIM TO PRIORITY

This application claims the benefit of U.S. Provisional Patent Application Ser. No. 63/557,640 filed Feb. 26, 2024, the entirety of which is incorporate herein by reference.

### BACKGROUND

### Technical Field

The embodiments herein generally relate to systems, methods, and mechanisms for injectable biomaterial-based therapeutic solutions, and in particular, to smart injectable mechanisms and systems that facilitate in situ scaffold formation, tissue regeneration, and organ repair through controlled transformations.

### Description of the Related Art

Tissue engineering and regenerative medicine have emerged as revolutionary fields in modern healthcare, addressing the critical need for effective repair and replacement of damaged or diseased tissues. Traditional approaches, including organ transplants and synthetic implants, often face limitations such as immune rejection, limited donor availability, and suboptimal integration with the host environment. Recent advancements in biomaterials and bio-fabrication techniques have provided a way for more sophisticated and patient-specific therapeutic solutions.

Biomaterials have gained significant attention due to their ability to conform to complex anatomical structures and provide a supportive microenvironment for cellular growth and tissue regeneration. These materials may be delivered in a minimally invasive manner and subsequently undergo in situ transformation into structured, functional implants that mimic biological and mechanical properties of native tissues. Such therapeutic systems offer numerous advantages, including reduced surgical complexity, enhanced biocompatibility, and the ability to incorporate bioactive agents that promote healing and regeneration.

Early-generation biomaterials primarily relied on pre-gelled or hydrogel formulations that required surgical placement or harsh cross-linking conditions for transformation. These limitations restricted their applicability to superficial wounds or surgically exposed tissues. Moreover, these mechanisms do not provide a hyper-customized or even reasonably customized mechanism for tissue growth and regeneration.

One of the key challenges has been developing therapeutic mechanisms that are injectable as well as smart and customized for specific individuals and bodily tissues. Moreover, despite the recent advancements, existing solutions still face challenges related to controlled degradation, mechanical stability, and efficient integration with host tissues. There is a need for an arrangement that addresses these limitations by introducing a smart injectable therapeutic mechanism and related systems that facilitate tissue generation, regeneration, repair, or growth and growth through minimally invasive methods.

### SUMMARY

An embodiment herein provides a therapeutic mechanism for tissue generation, regeneration, repair, or growth and therapeutic monitoring. The therapeutic mechanism may include a biocompatible ink configured to transition from an initial state to a structured form in response to interaction with predefined physiological biochemical, hormonal, or drug stimuli, and/or external stimuli post injection, such as into a target recipient. The therapeutic mechanism may include one or more therapeutic agents and a bio-sensing matter that may be configured to detect at least one biological parameter of the target recipient post transition of the biocompatible ink into the structured form.

An embodiment herein provides a therapeutic mechanism for tissue generation, regeneration, repair, or growth and therapeutic monitoring. The therapeutic mechanism may include configuring a biocompatible ink to transition from an injectable state to a structured form in response to interaction with predefined physiological and/or external stimuli post injection, such as into a target recipient. The biocompatible ink may be admixed with one or more regenerative agents and a bio-sensing matter. The bio sensing matter may be configured to detect at least one biological parameter of the target recipient post transition of the biocompatible ink into the structured form. The bio-sensing matter may include a nanostructure that may be configured to exhibit a change in properties upon interaction with the at least one biological parameter. The therapeutic mechanism may include administering the biocompatible ink to the target recipient. The therapeutic mechanism may include detecting the change in the properties using an external sensing device.

An embodiment herein provides a method of tissue generation, regeneration, repair, or growth and therapeutic monitoring with a therapeutic mechanism. The method may include selecting a biocompatible ink from a library of biocompatible inks. The library may include inks with predefined properties including viscosity, crosslinking behavior, biodegradability, mechanical strength, flexibility, mobility, cellular compatibility, hypoimmunogenic, or bio-identical and compatibility with bioactive agents. In embodiments, selecting the biocompatible ink may be based on a physiology of a target recipient for receiving the therapeutic mechanism. The method may include administering the selected biocompatible ink to the target recipient. The method may include causing the selected biocompatible ink to structurally and chemically undergo a transition into a structured form, such that the biocompatible ink defines and constitutes a substantial body portion of the therapeutic mechanism post-transition. In embodiments, causing the structural and chemical transition may be a result of one or more interactions at a target site of the target recipient including biomolecular interaction, hydrophobic interaction, hydrogen bonding, ionic interaction, or exposure to polyvalent ions.

### BRIEF DESCRIPTION OF THE DRAWINGS

The features of the disclosed embodiments may become apparent from the following detailed description taken in conjunction with the accompanying drawings showing illustrative embodiments herein, in which:

FIG. 1 illustrates a therapeutic mechanism, in accordance with some embodiments.

FIG. 2 illustrates an exemplary embodiment of transformation of the therapeutic mechanism from an initial state to a structured form after administering to a target site within a target recipient.

FIG. 3 illustrates an exemplary representation of the therapeutic mechanism post administration and transformation at the target site.

FIG. 4 illustrates administration of the therapeutic mechanism to the target site within the target recipient using an injection device, in accordance with some embodiments.

FIG. 5 illustrates administration of the therapeutic mechanism to the target site within the target recipient using an injection device, in accordance with some embodiments.

FIGS. 6-9 illustrate exemplary embodiments of a structured form achieved following transformation of a biocompatible ink of the therapeutic mechanism.

FIG. 10 illustrates a representation illustration of the transformation process of the biocompatible ink into the structured form at the target site, in accordance with some embodiments.

FIG. 11 illustrates sensing matter of the therapeutic mechanism, in accordance with some embodiments.

FIG. 12 illustrates administration of the therapeutic mechanism to the target site within the target recipient using a 3D printer, in accordance with some embodiments.

FIG. 13 illustrates an exemplary system for customizing and/or selecting the biocompatible ink from a library of biocompatible-inks, in accordance with some embodiments.

FIG. 14 illustrates the therapeutic mechanism configured for dental tissue generation, regeneration, repair, or growth and support, upon transitioning into the structured form, in accordance with some embodiments.

FIG. 15 illustrates the therapeutic mechanism configured to support or generate a cardiovascular tissue, upon transitioning into the structured form, in accordance with some embodiments.

FIG. 16 illustrates a representative hardware environment for practicing various embodiments.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In the following detailed description, reference is made to the accompanying drawings which form a part hereof, and in which is shown by way of illustration specific embodiments in which the embodiments herein may be practiced. These embodiments, which are also referred to herein as “examples,” are described in sufficient detail to enable those skilled in the art to practice the embodiments herein, and it is to be understood that the embodiments may be combined, or that other embodiments may be utilized and that structural, logical, and electrical changes may be made without departing from the scope of the embodiments herein.

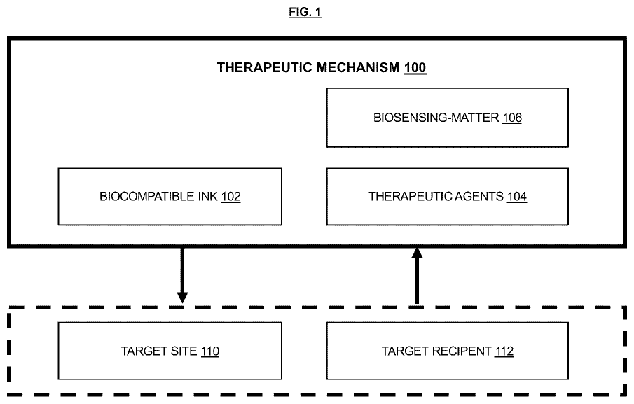

FIG. 1 illustrates a therapeutic mechanism 100, in accordance with various embodiments. The therapeutic mechanism 100 may be engineered for, among other things, tissue generation (and regeneration referred interchangeably to mean both, unless otherwise clear from the context), tissue repair, therapeutic delivery, bio-sensing, and the like. In example embodiments, being engineered may include physical, logical, theoretical, and other types of engineering activities and the like. In this context, being engineered for a purpose may also include being configured for such a purpose, being defined for such a purpose, being adapted for such a purpose, and the like. The therapeutic mechanism 100 may include a composition in an administrative state, such as an injectable state, a 3d printable state, an infusible state, a digestible state, and the like. The composition may include and/or be composed of a biocompatible ink 102. The therapeutic mechanism 100 may include one or more therapeutic agents 104, a bio-sensing matter 106, and other solutions, compositions, ingredients, cells, and the like. The various constituents of the therapeutic mechanism 100 may be configured to achieve, among other things, a synergistic functional integration within an internal physiological environment of a recipient (herein generally referred to as a “target”). In example embodiments, the therapeutic mechanism 100 is deployed or delivered or implanted or printed (referred herein interchangeably based at least on context, but in cases without limitations), thereby enabling structural, therapeutic, and functional efficacy in response to administration of the therapeutic mechanism 100 in a target environment, such as inside the body of a target recipient 112 at a target site 110. In embodiments, the biocompatible ink 102 may be configured to support the therapeutic agents 104, as a by being configured as and/or configured to provide a biocompatible matrix that may provide structural and functional support to the therapeutic agents 104. The biocompatible matrix may serve as a carrier, encapsulating the therapeutic agents 104 to protect them from premature degradation, control their release kinetics, and facilitate their targeted delivery at the target site 110, among other things. For example, the biocompatible matrix may allow sustained release of growth factors over time, stabilize bioactive proteins to maintain their efficacy, or improve cellular uptake of regenerative agents by providing a conducive microenvironment. The biocompatible matrix may be one of several different types of matrices either of a single formulation or of a blend of formulations, depending on interactions with one or more regenerative agents.

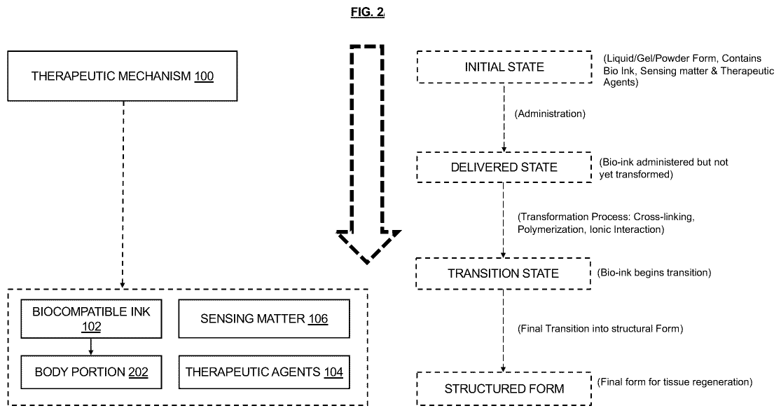

The biocompatible ink 102 may constitute and/or convey a primary structural framework of the therapeutic mechanism 100 and may be chemically and/or physically formulated to transition from its initial (e.g., injectable) state to a structured form, as shown in FIG. 2, such as in response to an interaction with physiological stimuli, such as predefined stimuli inside the body of the target recipient 112 post deployment. These stimuli may be present in and/or intrinsic to internal milieu of the target recipient's body and may include, but are not limited to, variations in pH, ionic concentrations, enzymatic activity, temperature, and the like. In example embodiments, portions of these stimuli may be included as part of an administrative plan, such as by being administered with or contemporaneously with the biocompatible ink 102. Further agents that influence activity of these stimuli may be similarly part of an administrative plan. In example embodiments, a first set of further agents may activate one or more stimuli, while another set of further agents may diminish activity of one or more other stimuli for, among other things, configuring a suitable in vivo environment. This target 112 location-based (e.g., in-situ) state transition of the biocompatible ink 102 may provide an ability to form a cohesive and stable structure in support of at least a portion of the therapeutic mechanism 100, which may be configured to anchor and/or sustain integration of other constituents within a physiological environment of the target's anatomy. Anchoring may include chemical or other bonding to one or more types of tissue in proximity to the target site 110. Anchoring may include at least partially stable placement at the target site 110. Anchoring may also include configuring a structure that interlocks with anatomical structures, blood vessels, arteries, cartilage, and the like. The biocompatible ink 102 may be configured to interact chemically, biologically, physically, temporally, invasively, electrochemically, magnetically, thermally, biologically, and/or structurally with one or more predefined physiological and/or external stimuli, such as described herein within the target 112, post administration.

In various embodiments, the initial state may include a liquid, semi-liquid, gel, powdered, or other flowable or printable, depositable state without limitations, or combinations thereof, allowing for controllable delivery of the therapeutic mechanism 100 to the target site 110 within the target recipient 112. In example embodiments, the initial state may include a series of different types of state selected from the list above, provided during administration. In an example, the therapeutic mechanism 100 may initially be administered as a liquid, followed at least in part by a gel, and further administered as liquid. An order or types of initial state, a timing of delivery of each type of initial state, concentrations, quantities, and the like of each type of initial state may be adapted based on aspects of the therapeutic mechanism 100, a desired final structured form, a target therapeutic objective, and the like. For example, when targeting cartilage repair, an initial liquid phase may deliver growth factors, followed by a gel that forms a supportive scaffold, with a final liquid phase containing anti-inflammatory agents to aid recovery.

In some embodiments, the structured form post transition may be a solidified form. In some embodiments, the structured form may be a gel form, which may be configured to provide, among other things, a hydrated, viscoelastic matrix for cellular integration and tissue regeneration. In some embodiments, the structured form may be a porous form, allowing for cellular infiltration, vascularization, and extracellular matrix deposition. In some embodiments, the structured form may be a fibrous network, mimicking an extracellular matrix for cellular attachment and growth. In some embodiments, the structured form may be a layered composite structure. In some embodiments, the structured form may be a biodegradable mesh, providing temporary structural support while facilitating tissue regeneration and gradual degradation. In various embodiments, the structured form may be configured to mimic a portion of human anatomy. In example embodiments, to mimic may include “bio-twinning,” optionally with corresponding (e.g., identical) physiologic function. The structured form may be configured to integrate with a portion of the human anatomy of the target recipient 112 for which the biocompatible ink 102 is targeted, and may include scaffolding for biocompatible coatings to include endogenous and/or exogenous cells and cell lines to confer specific desired biological and biophysical properties, and the like. To integrate with may also include co-existence while sharing at least a commonality of tissue structure, composition, biology, and the like at least in part, such as at an outer region/surface of the structured form.

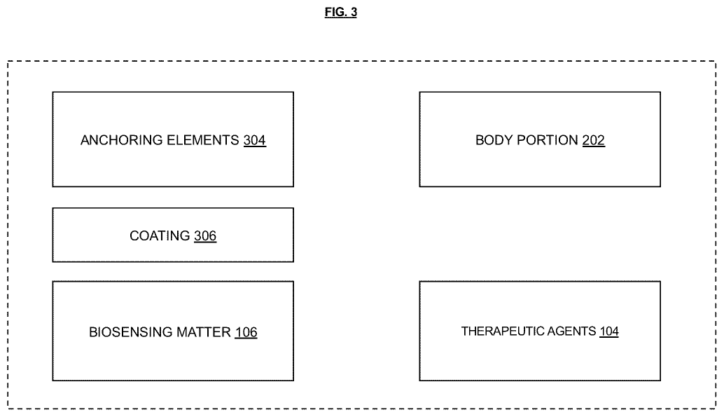

The biocompatible ink 102, which may be an injectable combination of substances, may also be referred to as bio ink 102 interchangeably without limitations. This biocompatible ink 102 may be configured to be administered using an injection device, such as a syringe 402, catheter, or a minimally invasive delivery system, as is described elsewhere herein and/or known in the art. Once administered, portions of the therapeutic mechanism 100 may undergo one or more transformations. In embodiments, the biocompatible ink 102 may be structurally and chemically configured to undergo the transition into the structured form, such that the biocompatible ink 102 defines and constitutes at least a precursor to a substantial body portion 202 (as exemplarily shown in FIGS. 2 and 3) of the therapeutic mechanism 100 post-transition.

In various embodiments, the biocompatible ink 102 may be made of a biodegradable material that may be configured to degrade at a controlled rate corresponding to tissue generation. The biodegradable material may be safe for use with human anatomy, and the like The rate of degradation may be controlled based on a range of factors including a target therapeutic effect, an administration plan, a doctor's preference, biological factors associated with the target recipient 112, and the like. The biodegradable material may be composed of one or more biomaterials such as a polymer, hydrogel, or a bioactive ceramic, without limitations. In embodiments, the biocompatible ink 102 may include one or more of nanoparticles, natural polymers, synthetic polymers, ceramics, bioceramics, composites, metals, hydrogels, genetically modified materials, polymeric nanocomposites, self-assembling materials, sol-gels, hybrid organic-inorganic materials, magnetic materials, conductive materials, cell laden materials, graphene, carbon-based materials, and the like without limitations. In some embodiments, the bio ink 102 may comprise various types of polymers without limitations such that, upon transition, the resulting structured form may constitute a bio-polymeric substrate, synthetic biomaterials, natural-derived biomaterials, and combinations thereof.

In examples, the bio ink 102 may include an injectable hydrogel. The injectable hydrogel may include a cross-linkable macromolecular network that may retain a high water content and may undergo in situ gelation upon administration. The gelation may be triggered by physiological cues such as temperature, pH, ionic strength, light, enzyme, or a biomolecular interaction and the like without limitations at the target site 110. This may form a structurally stable scaffold with predefined desired biomechanical and/or biochemical properties.

In examples, the bio ink 102 may include peptide-amphiphile (PA) molecules that may self-assemble into a biomimetic nanofibrous supramolecular architecture upon exposure to physiological conditions within the target recipient 112. The self-assembly of the PA molecules may be driven by a hydrophobic interaction, a hydrogen bonding, or an ionic interaction, and the like without limitations. The transformation may occur in response to a pH change, forming a structurally stable or bioactive scaffold for tissue regeneration in examples.

In examples, the bio ink 102 may include an injectable composition that may include peptide-amphiphile (PA) molecules that may remain as amorphous aggregates at neutral pH. The PA molecules may undergo self-assembly into cylindrical micelles upon exposure to polyvalent ions. The polyvalent ions may neutralize electrostatic repulsion, facilitating physical crosslinking and the transformation to create a structurally stable scaffold for tissue regeneration. The transformation of the injectable composition into the structured form may be facilitated by a mechanism selected from polymerization, precipitation, cross-linking and the like without limitations induced by ionic interactions or thermal processes, forming a structurally stable body portion of the therapeutic mechanism 100 for tissue integration and generation.

In various embodiments, the therapeutic mechanism 100 may facilitate target recipient-based in vivo 3D printing of the structured form. The therapeutic mechanism 100 may facilitate repair, growth, regrowth, generation, regeneration of bodily tissues and/or various anatomical structures or combinations thereof within the target recipient 112. The process of generation may include regeneration of a damaged portion of the recipient's body. In embodiments, the process of tissue generation may include in vivo regeneration. In various embodiments, the process of generation may include generation and/or may support generation of one or more of a cardiac valve, an arterio cruciate ligament, a knee joint, a hip joint, a shoulder joint, an intervertebral disc, meniscus tissue, a femur, a tibia, a spinal vertebra, a mandible (jawbone), an anterior cruciate ligament (ACL), a rotator cuff tendon, an Achilles tendon, a heart valve, a component of a prosthetic heart valve (e.g., a bioscaffold and/or an intracellular matrix), a coronary artery, a component of an artificial coronary artery or other vascular arterial structure (e.g., cerebral vascular, peripheral vascular, and the like), structures (e.g., arterial, venous, and lymphatic structures and vessels), vascular patches for coronary and peripheral vascular surgeries, a vascular graft, a peripheral nerve, an optic nerve, a retinal layer, a liver lobule, a pancreatic islet, a kidney nephron, a skeletal muscle tissue, a cardiac muscle tissue, cardiac muscle/myocardial tissue, endocardiu and pericardium (bioscaffold, intracellular matrix), a smooth muscle tissue, a component of smooth muscle tissue comprising cardiac, vascular, gastrointestinal, or urologic tissues (bioscaffold, intracellular matrix), a fascia tissue, an epidermal skin tissue, a dermal fibroblast, adipose tissue (e.g., subcutaneous fat tissue), an esophageal tissue, an intestinal villi, a bladder epithelium, a ureteral tissue, an endometrial tissue, an ovarian follicular tissue, testicular seminiferous tubules, a dentin tissue, a periodontal ligament, a nasal cartilage, a hyaline cartilage, an elastic cartilage, a fibrocartilage, a hepatic tissue, a biliary epithelium, pancreatic islet cells, exocrine pancreatic tissue, renal parenchymal tissue, glomerular structures, a renal tubular epithelium, a cortical neuronal tissue, a spinal cord tissue, a peripheral nerve tissue (e.g., by targeting applicable support cells), a retinal neural tissue, and the like without limitations. In example embodiments, the therapeutic mechanism 100 may facilitate kidney nephron replacement. In example embodiments, the therapeutic mechanism 100 may facilitate 3D printing utilizing the biocompatable ink (e.g., alone) to create a prosthetic, implantable (surgical and trans-catheter) cardiac valve, a scaffold of a bioprosthetic valve (e.g., when combined with specific biologic agents or biochemical modifications) or the like. In example embodiments, 3D printing may include utilizing the biocompatable ink to create a scaffold of a bioprosthetic valve that may be combined with specific cell coatings, and the like such as stem cells. In example embodiments, the methods and systems described herein may include bioprinting a scaffold seeded with stem cells that would then be implanted as a functional valve.

In example embodiments, the therapeutic mechanism 100 may be configured to improve and/or facilitate resistance to thrombosis. The therapeutic mechanism 100 may include Magnetic Resonance Imaging (MRI) and electromagnetic field compatibility.

In example embodiments, the therapeutic mechanism 100 may include an ability to replicate in vivo dimensions based on external imaging “twinning” such as based on CT or MRI data. The therapeutic mechanism 100 may also be configured to replicate or replace an in vivo biological structure in terms of physiologic and biophysical properties. The therapeutic mechanism 100 may interact or combine with other biologic structures (cells) or substances to enhance a final structure's biomechanical or biophysical properties. Also, the therapeutic mechanism 100 may serve as a substrate or foundation for integration with selected cell lines or tissues to replicate or replace a biological structure in terms of function and physiology.

In various embodiments, the therapeutic mechanism 100 may include the one or more therapeutic agents 104 that may be configured to facilitate treatment, healing, regeneration, or functional restoration of tissues or organs or bones generally proximal to the target site 110. In various embodiments, the therapeutic agents 104 may be admixed within the biocompatible ink 102. In various embodiments, the therapeutic agents 104 may be administered according to an administration plan that may include sequential delivery of; a portion of the biocompatible ink 102, a portion of the therapeutic agents 104 and the like in any order and in any combination. A therapeutic mechanism 100 administration plan may be determined based on a range of factors that may include administration factors such as but not limited to type and severity of the condition being treated, physiological characteristics of the target site 110 (e.g., tissue type, vascularization, mechanical properties), target-specific factors (e.g., age, immune response, metabolic rate), or a desired therapeutic outcome (e.g., controlled release, rapid integration, or sustained regeneration).

In embodiments, the therapeutic agents 104 may be embedded within the biocompatible ink 102 in various forms such as but limited to encapsulated in microspheres, conjugated to a biopolymeric substrate, or suspended in the bio-ink. In certain embodiments, the therapeutic agents 104 may include anti-inflammatory agents, analgesic agents, antimicrobial agents, growth factors, regenerative agents, angiogenic or anti-angiogenic agents, immunomodulatory agents, anti-cancer agents, osteogenic or chondrogenic agents, neuromodulatory agents, anticoagulants, wound healing and fibrosis-modulating agents, endocrine modulators, gene therapy agents, biopolymer-based drug delivery systems, and bioelectric or biophysical stimulation agents.

In various embodiments, the therapeutic agents 104 may include anti-inflammatory agents such as but not limited to non-steroidal anti-inflammatory drugs (NSAIDs), such as ibuprofen, naproxen, or celecoxib, as well as corticosteroids, including dexamethasone, prednisolone, or hydrocortisone, and the like without limitations. Dosage, activation modes, and admixed variants may be determined and/or adapted based on prior effectiveness measures, target recipient biology/anatomy/physiology, and the like. In some embodiments, biologic anti-inflammatory agents, such as tumor necrosis factor (TNF) inhibitors, interleukin inhibitors, or monoclonal antibodies, may be utilized in the therapeutic agents 104 to modulate an inflammatory response at the target site 110. In some embodiments, the therapeutic agents 104 may include analgesic agents, such as but not limited to opioid analgesics such as morphine, fentanyl, or oxycodone, non-opioid analgesics such as acetaminophen or aspirin, or local anesthetics such as lidocaine or bupivacaine, to alleviate pain associated with tissue injury or implantation.

The therapeutic agents 104 may include one or more portions of the regenerative agents within the composition of the biocompatible ink 102. The regenerative agents may include but are not limited to stem cells, bioactive proteins, differentiated cell lines and/or pleuripotential stem cells, or therapeutic compounds exhibiting regenerative bioactivity. The regenerative agents may be embedded within and/or administrated with the biocompatible ink 102 to ensure controlled release and/or targeted therapeutic effect. The stem cells may include multipotent or pluripotent lineages capable of differentiating into requisite tissue types, while the bioactive proteins may include growth factors or signaling molecules that facilitate tissue repair, cellular proliferation, and angiogenesis. In accordance with various embodiments, the therapeutic agents 104, in this context, may refer to molecules that have an ability to improve tissue regeneration or mitigate pathological conditions through localized pharmacological action.

In various embodiments, the therapeutic agents 104 may include antibiotics and/or antimicrobial agents configured to prevent or mitigate infections. The antimicrobial agents may include broad-spectrum antibiotics, such as penicillins, cephalosporins, or fluoroquinolones, as well as antifungal agents such as amphotericin B or fluconazole, and/or antiviral agents such as acyclovir or remdesivir. In certain embodiments, antiseptics, such as chlorhexidine or silver nanoparticles, may be incorporated within the therapeutic agents 104 to provide localized antimicrobial activity.

In various embodiments, the therapeutic agents 104 may include growth factors to improve tissue regeneration and/or repair. The growth factors may include, but are not limited to, epidermal growth factor (EGF), fibroblast growth factors (FGFs), vascular endothelial growth factor (VEGF), bone morphogenetic proteins (BMPs), transforming growth factor-beta (TGF-B), or platelet-derived growth factor (PDGF). In some embodiments, the regenerative agents such as mesenchymal stem cells (MSCs), induced pluripotent stem cells (iPSCs), hematopoietic stem cells (HSCs), extracellular matrix components such as collagen, laminin, or fibronectin, or peptide-based regenerative agents such as RGD peptides may be utilized to improve tissue regeneration and/or repair.

In some embodiments, the therapeutic agents 104 may include angiogenic agents such as VEGF, basic fibroblast growth factor (bFGF), or angiopoictins to promote vascularization, while anti-angiogenic agents such as bevacizumab, endostatin, or thalidomide may be utilized to inhibit unwanted angiogenesis. In some embodiments, immunomodulatory agents such as cyclosporine, tacrolimus, azathioprine, interferons, interleukin-2 (IL-2), or granulocyte-macrophage colony-stimulating factor (GM-CSF) may be included to regulate immune responses at the target site 110.

In certain embodiments, the therapeutic agents 104 may include anti-cancer agents such as doxorubicin, cisplatin, paclitaxel, tyrosine kinase inhibitors, or monoclonal antibodies to inhibit tumor growth and progression.

In some embodiments, the therapeutic agents 104 may include osteogenic and chondrogenic agents to promote bone or cartilage formation. The osteogenic and chondrogenic agents may include BMPs, parathyroid hormone (PTH), calcitonin, TGF-β3, SOX9, or hyaluronic acid. In some embodiments, the therapeutic agents 104 may include neuromodulatory agents, such as but not limited to neurotrophic factors such as brain-derived neurotrophic factor (BDNF) or nerve growth factor (NGF), neuroprotective agents such as memantine and riluzole, or neurotransmitter modulators such as dopamine agonists or serotonin modulators, to facilitate nerve regeneration and functional restoration.

In some embodiments, the therapeutic agents 104 may include anticoagulants such as heparin, warfarin, or dabigatran, as well as thrombolytic agents such as tissue plasminogen activator (tPA) or streptokinase, that may be utilized to modulate clotting mechanisms. In some embodiments, the therapeutic agents 104 may include wound healing or fibrosis-modulating agents such as fibroblast growth factors (FGFs), PDGF, pirfenidone, or nintedanib to regulate scar formation and extracellular matrix remodeling. In some embodiments, the therapeutic agents 104 may include metabolic hormones such as insulin or glucagon-like peptide-1 (GLP-1) analogs, thyroid hormones such as levothyroxine or liothyronine, and/or sex hormones such as estrogen, testosterone, or progesterone.

In some embodiments, the therapeutic agents 104 may include gene therapy agents to modify cellular behavior at the target site 110. The gene therapy agents may include plasmid DNA, CRISPR-Cas9 constructs, small interfering RNA (siRNA), messenger RNA (mRNA), or microRNA, without limitations. In some embodiments, the therapeutic agents 104 may include biopolymer-based and smart drug delivery systems, including hydrogels such as alginate and hyaluronic acid hydrogels, nanoparticle-based carriers such as liposomes or polymer-based nanocarriers, or microspheres composed of poly (lactic-co-glycolic acid) (PLGA) or chitosan without limitations.

In various embodiments, the therapeutic agents 104 may include bioelectric or biophysical stimulation agents that may be included to improve tissue regeneration and/or functional recovery. The bioelectric or biophysical stimulation agents may include such as but not limited to electrostimulation-based bioelectric signaling modulators or magnetically responsive nanoparticles such as superparamagnetic iron oxide nanoparticles (SPIONs), and the like. The selection, concentration, and mode of delivery of the therapeutic agents 104 may be customized based on physiological parameters of the target 112, an intended therapeutic effect, or biophysical properties of a delivery system 406 utilized therein. In various embodiments, the therapeutic agents 104 may be incorporated into injectable biomaterials, bio-inks, nanocarriers, or scaffold-based systems to improve their efficacy and/or controlled release.

In various embodiments, the therapeutic agents 104 may be dispersed within the biocompatible ink 102 such that the therapeutic agents 104 may include at least one of stem cells, progenitor cells, cytokines, extracellular vesicles, bioactive peptides, growth factors, bioactive proteins, or therapeutic compounds exhibiting regenerative bioactivity and configured to promote tissue generation at the target site 110. The therapeutic agents 104 may be released in a controlled manner post-implantation or deployment at the target site 110. In some embodiments, the therapeutic agents 104 may include mesenchymal stem cells (MSCs), induced pluripotent stem cells (iPSCs), or embryonic stem cells (ESCs) encapsulated within the biocompatible ink 102 to facilitate cellular differentiation and/or tissue generation. In some embodiments, the therapeutic agents 104 may include exosomes derived from the stem cells. The exosomes may contain regenerative biomolecules including such as microRNAs, proteins, or lipids and the like to modulate cellular repair and/or generation. In some embodiments, the therapeutic agents 104 may include at least one of platelet-derived growth factor (PDGF), vascular endothelial growth factor (VEGF), fibroblast growth factor (FGF), bone morphogenetic proteins (BMPs), or transforming growth factor-beta. In some embodiments, the therapeutic agents 104 may be encapsulated within micro- or nano-carriers embedded in the biocompatible ink 102. The micro- or nano-carriers may be selected from liposomes, polymeric microspheres, hydrogel nanoparticles, or dendrimers. The micro-or nano-carriers may be configured to provide controlled, sustained, or stimuli-responsive release of the therapeutic agents 104 at the target site 110.

In various embodiments, the therapeutic mechanism 100 is designed in such a way that the body portion 202 after the transition provides a controlled, sustained release of the therapeutic agents 104 including such as immune-modulating agents, including one or more of cytokines, checkpoint inhibitors, monoclonal antibodies, or growth factors and the like as discussed elsewhere in the document without limitations. In embodiments, the body portion 202 may be configured to activate and recruit immune cells at the target site 110 of the target 112, promoting a localized immune response, and supporting tissue generation proximate to the body portion 202 of the therapeutic mechanism 100.

In some embodiments, the body portion 202 of the therapeutic mechanism 100 may include a biodegradable scaffold that may gradually resorb over time while being replaced by or integrated into native tissue. In some embodiments, the body portion 202 of the therapeutic mechanism 100 may include an extracellular matrix-derived scaffold. The extracellular matrix-derived scaffold may be selected from collagen, fibrin, chitosan, laminin, proteoglycans, hyaluronic acid, or combinations thereof, in various embodiments, without limitations.

In embodiments, the body portion 202 of the therapeutic mechanism 100 may include a hydrogel-based matrix that may be configured to undergo a controlled swelling response to physiological conditions for modulating one or more mechanical properties or biochemical properties post-implantation into the target recipient 112.

In embodiments, the body portion 202 of the therapeutic mechanism 100 may include a ceramic-reinforced composite. The ceramic-reinforced composite may include at least one of hydroxyapatite, tricalcium phosphate, or bioactive glass, zirconia-reinforced lithium silicate, and the like without limitations, to enhance osteointegration in bone tissue applications.

In embodiments, the body portion 202 of the therapeutic mechanism 100 may include a nanostructured scaffold. The nanostructured scaffold may have a surface topology that may be engineered to enhance cellular adhesion, migration, or differentiation, and the like without limitations.

In embodiments, the body portion 202 of the therapeutic mechanism 100 may include a multi-layered structure. The multi-layered structure may include at least one inner support layer providing mechanical stability. The multi-layered structure may include an outer bioactive layer that may facilitate promoting cellular interaction and integration.

In embodiments, the body portion 202 of the therapeutic mechanism 100 may be mechanically tunable, such that one or more mechanical properties of the body portion 202 may be adjustable by modifying one or more of polymer crosslinking density, degradation rate, or hydration capacity, and the like without limitations.

In embodiments, the body portion 202 of the therapeutic mechanism 100 may be configured to exhibit shape memory properties. The shape memory properties may enable the therapeutic mechanism 100 to conform dynamically to tissue defects upon interaction with the predefined physiological and/or external stimuli.

In embodiments, the body portion 202 of the therapeutic mechanism 100 may include a biocompatible scaffold. The biocompatible scaffold may be configured to facilitate tissue generation by supporting cell adhesion, proliferation, or differentiation for construction of one or more of a bone, cartilage, soft tissue, neural tissue, and a vascular structure. The biocompatible scaffold may be configured to provide a controlled release of one or more of therapeutic, regenerative, immunomodulatory, or anti-cancer agents for localized treatment. The biocompatible scaffold may be configured to modulate immune response so that the biocompatible scaffold may promote immune activation for cancer immunotherapy, immune suppression for transplantation tolerance or autoimmune disease management and the like without limitations. The biocompatible scaffold may be configured to improve angiogenesis or vascularization by promoting formation and/or growth of new blood vessels for improved integration with a host tissue into the target recipient 112. The biocompatible scaffold may be configured to incorporate bioactive molecules, bioactive proteins, stem cells, growth factors, or therapeutic compounds and the like that may exhibit regenerative bioactivity to facilitate functional recovery and a tissue engineering application among various other applications without limitations. The biocompatible scaffold may be configured to serve as a therapeutic platform for an oncology application. In such situations, the scaffold may be configured to deliver chemotherapeutic or immunotherapeutic agents to a localized tumor site. The biocompatible scaffold may be configured to facilitate modulate a tumor microenvironment to improve immune cell infiltration or target cancer cells via selective apoptosis-inducing biomaterials. The biocompatible scaffold may be configured to provide a biomechanical support by maintaining shape or mechanical stability at the target site 110. The biocompatible scaffold may include a biomaterial designed to mimic mechanical properties of a supported tissue at the target site 110.

In embodiments, the bio-sensing matter 106 may be integrated into, and or administered with the biocompatible ink 102. The bio-sensing matter 106, which may be configured as a constituent of the therapeutic mechanism 100, may be homogenously admixed within the biocompatible ink 102 in various embodiments. The bio-sensing component may be composed of a material engineered to detect and/or monitor a plurality of biological parameters, optionally in real time. In example embodiments, the bio-sensing matter 106 may facilitate detection and/or monitoring of the plurality of biological parameters, such as by a separate device, mechanism, or the like. These biological parameters may include, but are not limited to, hemodynamic and/or vascular flow characteristics, tissue oxygenation, systemic or localized glucose concentrations, or biochemical variations indicative of pathological or metabolic processes. After and/or during the in-situ state transition of the biocompatible ink 102, the bio-sensing component may spatially integrate with a resulting transformed and structured body portion 202 of the biocompatible ink 102, thereby establishing functional connectivity with surrounding biological tissues. The functional connectivity may enable the bio-sensing component to operate as a localized diagnostic interface, providing actionable and/or measurable insights into a dynamic physiological state of a proximate environment of the administered therapeutic mechanism 100.

These unique configurations and interplay of various elements of the therapeutic mechanism 100 with the target anatomy may allow the therapeutic mechanism 100 to perform multifaceted roles, including the regeneration of a damaged tissue, localized delivery of the therapeutic agents 104, and monitoring of physiological parameters proximate to the structured form of the biocompatible ink 102 post transformation at the target site 110. These features collectively may render the therapeutic mechanism 100 particularly suitable for applications in regenerative medicine, personalized healthcare, and advanced therapeutic interventions.

FIG. 2 illustrates an exemplary embodiment of the transformation of the therapeutic mechanism 100 from the initial state to the structured form. Initially, the therapeutic mechanism 100 is in the administrative state such as the injectable state before delivery or deployment into the target site 110, such as within the body of the target recipient 112. As discussed above, the therapeutic mechanism 100 may include the biocompatible ink 102. Once the therapeutic mechanism is deployed into a delivered state, the bio-ink 102 may interact with the physiological stimuli. In response to the interaction of the biocompatible ink 102 with the physiological stimuli intrinsic to the target recipient's body, the biocompatible ink 102 may undergo the in-situ phase transition from the initial state such to the structured form, resulting in the formation of the body portion 202. The structured form may be achieved through a transition state wherein chemical or physical changes may start occurring in the therapeutic mechanism. The transition state may include such as without limitations solidification, cross-linking, polymerization, ionic interaction, and the like. The body portion 202 may serve as the scaffold for regenerative processes and may retain the embedded constituents such as the therapeutic agents 104 and/or the bio sensing matter 106 within its three-dimensional network, in some embodiments. In some embodiments, the bio sensing matter 106 and/or the therapeutic agents 104 may transform into the structured form along with the biocompatible ink 102. In some embodiments, one or both of the bio sensing matter 106 and the therapeutic agents 104 may not transform into the structured form but instead retain their original form and remain binding with the body portion 202 so as to release in a controlled manner in a desired manner.

In some embodiments, the therapeutic agents 104 may be dispersed within the biocompatible ink 102 in a manner that ensures their spatial availability and/or functional efficacy following the transformation. The therapeutic agents 104 may be strategically positioned to facilitate localized therapeutic effects and/or tissue regeneration.

The bio-sensing matter 106 may be uniformly integrated within the biocompatible ink 102. In its operative configuration, the bio-sensing matter 106 may establish functional interaction with the body portion 202, enabling detection and/or real-time monitoring of various biological parameters. The functional interaction of the bio-sensing matter 106 with the surrounding tissue may be achieved through spatial integration of the sensing matter 106 within the biocompatible ink 102 during and after the transformation.

FIG. 3 illustrates an exemplary block diagram of the therapeutic mechanism 100 post deployment and transformation at the target site 110. Referring to FIGS. 2 and 3, the therapeutic mechanism 100 is further described herein, in accordance with some embodiment. The biocompatible ink 102 post transition may form the body portion 202 as shown. In some embodiments, if the bio-ink 102 transitions into a scaffold, hydrogel, or fibrous matrix with inherent adhesion or integration properties, additional anchoring elements 304 (such as staples or sutures) may not be necessary. These materials may naturally adhere to surrounding tissues through biological interactions, cellular infiltration, or extracellular matrix deposition.

In embodiments, the body portion 202 of the therapeutic mechanism 100 that forms a substantial portion of the structured form of the biocompatible ink 102 may be configured to form a support structure to provide a desired tensioning and supportive force to a bodily tissue at or proximate to the target site 110 of the target recipient 112 such as to manage and/or treat incontinence, prolapse, hernia, heart tissue repair, and support or regenerate or repair various other bodily tissues, or treat or manage a variety of conditions without limitations.

In embodiments, the body portion 202 of the therapeutic mechanism 100 may be configured to form a scaffold at the target site 110 within the target recipient 112. In embodiments, the scaffold may be configured to provide necessary structural support to a bodily tissue, bone, skin tissue, soft tissue, cartilage, ligament, tendon, and interfaces of the aforementioned tissues, and the like, to facilitate cellular infiltration, vascularization, extracellular matrix deposition, or osteointegration for tissue regeneration and repair.

In embodiments, the scaffold may be configured to function as a controlled-release platform for the one or more therapeutic agents 104 (hereafter referred to as therapeutic agents 104 for simplicity of description without limitations).

In embodiments, the body portion 202 of the therapeutic mechanism 100 may be configured as a bioactive implant attached to a bodily issue or organ at the target site 110 within the target recipient 112. The bioactive implant may be designed to release the therapeutic agents 104 in a controlled manner to promote cellular proliferation, differentiation, and tissue regeneration.

In embodiments, the body portion 202 of the therapeutic mechanism 100 may be configured as a hydrogel matrix at the target site 110 within the target recipient 112. The hydrogel matrix may provide a hydrated, biocompatible microenvironment that supports cell adhesion, migration, or tissue remodeling while offering tunable mechanical properties and degradation rates tailored to specific therapeutic needs.

In embodiments, the body portion 202 of the therapeutic mechanism 100 may be configured as a cellularized construct at the target site 110 within the target recipient 112. The cellularized construct may incorporate autologous, allogeneic, or stem cells to enhance tissue regeneration by actively participating in extracellular matrix deposition and/or remodeling processes.

In embodiments, the body portion 202 of the therapeutic mechanism 100 may be configured as a composite graft at the target site 110 within the target recipient 112. The composite graft may integrate multiple material components, such as biopolymers, ceramics, or metallic reinforcements, to provide mechanical stability and bioactivity for applications such as bone repair, load-bearing tissues, tissue or bone growth, or joint resurfacing and the like without limitations.

In embodiments, the body portion 202 of the therapeutic mechanism 100 may be configured as a self-assembling nanofiber network at the target site 110 within the target recipient 112. The nanofiber network may be designed to mimic a native extracellular matrix, providing nanoscale topographical cues that guide cellular behavior and tissue morphogenesis.

In embodiments, the body portion 202 of the therapeutic mechanism 100 may be configured as a drug-eluting depot at the target site 110 within the target recipient 112. The drug-eluting depot may be designed to provide localized and sustained release of the therapeutic agents 104, such as anti-inflammatory drugs, antibiotics, or regenerative molecules, among others without limitations to modulate healing response and/or prevent complications.

In embodiments, the body portion 202 of the therapeutic mechanism 100 may be configured as a bioresorbable filler at the target site 110 within the target recipient 112. The bioresorbable filler may be used to temporarily occupy tissue voids, support surrounding structures, and/or gradually degrade as native tissue regenerates, reducing the need for secondary interventions.

In embodiments, the body portion 202 of the therapeutic mechanism 100 may include a drug-eluting implant that may be configured to provide localized and sustained release of the therapeutic agents 104 to the target site 110. The drug-eluting implant may be biodegradable or non-biodegradable and may be tailored for various medical applications, including wound healing, cartilage repair, tissue repair, or bone regeneration, and the like without limitations.

In embodiments, the body portion 202 of the therapeutic mechanism 100 may be applied a coating 306 such as a bioactive coating among various other types of coating 306 materials without limitations. The coating 306 that may be applied to improve biocompatibility, reduce inflammation, and/or promote cellular adhesion. The bioactive coating may be composed of proteins, peptides, or synthetic biomaterials that interact with a host tissue at the target site 110 to support healing and/or integration within the target recipient 112.

In embodiments, the body portion 202 of the therapeutic mechanism 100 may be configured as a bioprinted construct, such that the bioprinted construct is fabricated using 3D bioprinting technology to create customized anatomical structures that replicate patient-specific tissue architecture. The bioprinted construct may be designed to contain the therapeutic agents 104 to improve integration with surrounding tissues.

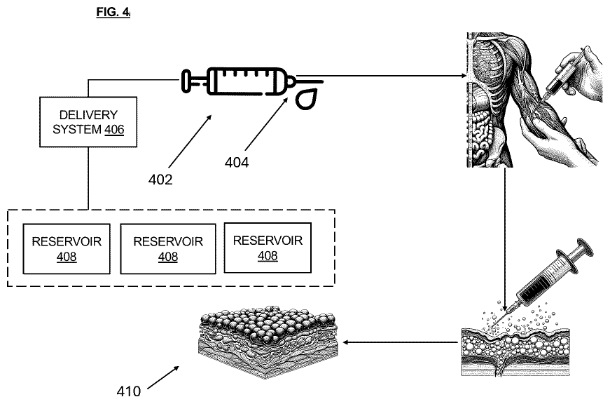

Referring now to FIG. 4, in some embodiments, the therapeutic mechanism 100 may be delivered to the target site 110 within the target recipient 112 using an injection device, such as a syringe 402 or any other suitable applicator configured for controlled administration without limitations. The injection device may be operable to dispense the biocompatible ink 102 from its initial state, which may include a liquid state, semi-liquid state, gel state, or any other injectable form suitable for delivery without limitations, as discussed elsewhere in the document. In certain embodiments, the injection device may include a needle 404, cannula, catheter, or any other conduit without limitations that may be capable of directing the biocompatible ink 102 to the target site 110 within the target recipient's body.

After administration of the biocompatible ink 102 through the injection device 402, the biocompatible ink 102 may undergo the transformation leading to formation of the structured form at the target site 110. In accordance with the embodiment illustrated in FIG. 4, following the transformation of the biocompatible ink 102 into its structured form, the body portion 202 of the biocompatible ink 102 may develop into a patch 410. The patch 410 may comprise a solid form, a structured form, a semi-solid form, an increased density region, a regular shape, an irregular shape, a target anatomy defined shape, uniform density, varying density, and the like.

The patch 410 may conform to anatomical contours of the target site 110 and may provide structural reinforcement, support, or a bioactive interface for tissue integration, healing, regeneration, or regrowth. In various embodiments, the patch 410 may exhibit bio-mechanical properties suitable for intended therapeutic application, including but not limited to elasticity (e.g., viscoelasticity), tensile strength, porosity, degradation kinetics, or bioactivity, and the like without limitations. In embodiments, the patch 410 may function as a temporary or long-term scaffold, facilitating cellular infiltration, vascularization, extracellular matrix deposition, or tissue remodeling, eventually leading to the regrowth or regeneration of native tissue at the target site 110. In embodiments, the patch 410 may function as a temporary or long-term implant for the treatment of a variety of conditions. In some embodiments, the patch 410 may be bioresorbable, that may gradually degrade over time in response to physiological conditions while being replaced by newly formed tissue. Degradation profile of the patch 410 may be tunable based on composition of the biocompatible ink 102, which may allow sustained therapeutic efficacy tailored to the specific application.

In embodiments, the transformation may involve the transition state during which the biocompatible ink 102 changes in viscosity, cross-links, polymerizes, precipitates, or otherwise solidifies based on physiological or externally applied stimuli as discussed in conjunction with FIG. 2 without limitations.

In embodiments, following injection of the biocompatible ink 102 into the target recipient 112 via the injection device, the biocompatible ink 102 may undergo the in situ transition from its initial state, which may be injectable without limitations in accordance with the embodiments shown in FIG. 4, to the structured form. The structured form may be adapted to provide a mechanical support, facilitate cellular integration, promote vascularization, or enable tissue regeneration at the target site 110. The transformation process may be tunable based on a variety of factors such as but not limited to a formulation of the biocompatible ink 102, selection of the therapeutic agents 104 incorporated therein, or environmental conditions at the target site 110, among many others.

In various embodiments, the injection device may be manually operated or incorporated into an automated or semi-automated delivery system 406 that may provide a controlled administration of the biocompatible ink 102 into the target recipient's body. In various embodiments, the injection device may further include one or more chambers or reservoirs 408a, 408b, 408c (also referred to as reservoir 408) for sequential or simultaneous delivery of different bio-ink 102 components, facilitating complex tissue engineering applications. The number of such reservoirs 408 shown in the FIG. 4 are merely for illustrative purpose and there could be more or less number of reservoirs 408 in various embodiments, In some embodiments, the injection device may be configured to deliver adjunct materials, such as cross-linking agents, stabilizers, or additional therapeutic agents 104, that may interact with the biocompatible ink 102 to modulate the transformation process or optimize formation of the structured form.

In embodiments, the delivery process, facilitated by the injection device, may provide a minimally invasive administration method while ensuring precise localization and/or controlled transformation of the therapeutic mechanism 100 to achieve an intended therapeutic outcome.

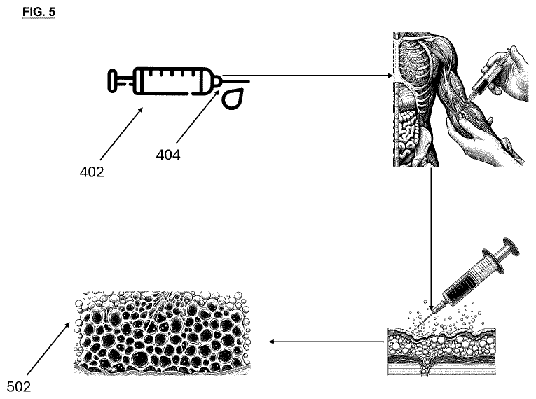

FIG. 5 illustrates an example of the therapeutic mechanism 100 that may be delivered to the target site 110 within the target recipient 112 using the injection device 402, such as the syringe or any other suitable applicator configured for precise administration without limitations similar to the injection device shown in FIG. 4. However, in accordance with the embodiments illustrated in FIG. 5, following the transformation of the biocompatible ink 102 into the structured form, the body portion 202 of the biocompatible ink 102 may develop into a mesh-based structure 502 (also referred to as a mesh) such as a mesh-based implant or another form of mesh-based body portion 202 including a mesh-based regenerated tissue and the like without limitations (hereafter referred to as a mesh 502).

In accordance with some embodiments, the mesh 502 may be implemented in slings, among various other forms without limitations, suitable for the treatment of male and female urinary or fecal incontinence such as to effect pelvic floor, perineal floor, or pelvic prolapse repairs employing a variety of surgical approaches. For example, female pelvic floor repair slings such as urinary slings or pelvic prolapse repair slings may be implanted by techniques that may involve transvaginal, transobturator, suprapubic, pre-pubic, or transperincal exposures or pathways, and male urinary incontinence slings may be implanted by techniques that may involve transobturator, suprapubic, or transperincal pathways. The disclosed embodiments may be used as fecal incontinence slings which may be implanted by techniques that may involve transvaginal, transobturator, suprapubic or via perineal floor pathways or through other methods or may be used for other uplift or reconstruction surgeries, without limitations.

In some embodiments, the mesh 502 may be used to suspend various bodily locations in the body of the target 112 such as pelvic organ of the recipient's body such as for the treatment of pelvic organ prolapse and the like without limitations. In some embodiments, the mesh 502 may be used in a urinary sling with some modifications. In some embodiments, the mesh 502 may be used in a retropubic incontinence sling. In some embodiments, the mesh 502 may be configured to be delivered by way of a transvaginal approach or a transobturator approach or vaginal pre-pubic approach or a laparoscopic approach or may be delivered through other methods and may be positioned at various locations within the recipient's body without limitations. In some embodiments, the mesh may be delivered through a sacrocolpopexy procedure.

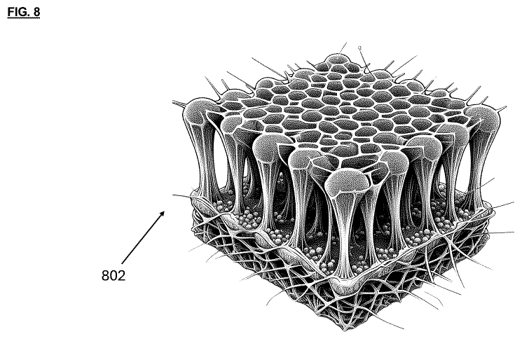

In various embodiments, the biocompatible ink 102, following the transformation into the structured form, may assume a variety of shapes and forms, including but not limited to scaffolds, implants, regenerated tissues, re-engineered tissues, regenerated bones, organ-like structures, tissue patches, cellular matrices, or hybrid composites and the like without limitations, incorporating one of both biological and synthetic components. The structured form may be configured as mesh-based structures such as the mesh 502, fibrous structures (such as fibrous structure 802 shown in conjunction with FIG. 8 later), woven structures, non-woven structures, microporous matrices, macroporous scaffolds, solid structures, semi-solid gel-like structures, stratified multilayered structures, lattice frameworks, hydrogel-based networks, nanofiber matrices, microcarrier-based assemblies, or bioactive coatings, and the like without limitations. In some embodiments, the structured form may be designed to mimic the extracellular matrix (ECM) of a specific tissue type, providing a biomimetic environment conducive to cell adhesion, proliferation, differentiation, vascularization, or tissue integration, and the like without limitations. In some embodiments, the structured form may exhibit controlled degradation properties, allowing for gradual replacement by native tissue over time, or may incorporate bioresorbable materials that may facilitate complete or partial assimilation into its surrounding biological environment. The structured form may include integrated reservoirs for sustained release of the therapeutic agents 104, growth factors, or cellular components to promote tissue regeneration and/and healing in a controlled and localized manner.

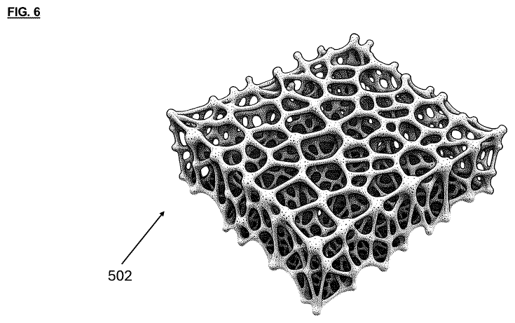

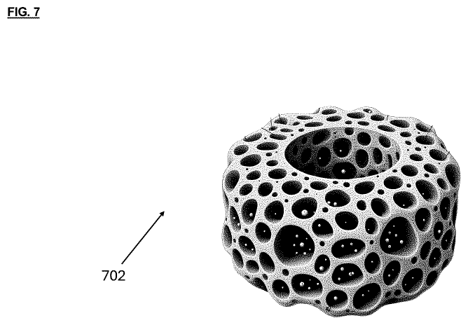

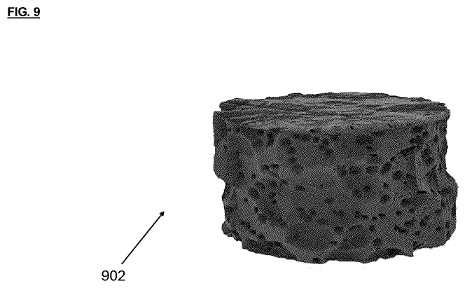

FIGS. 6-9 illustrate exemplary embodiments of the structured form achieved following the transformation of the biocompatible ink 102. The structured form may assume various shapes and configurations without limitation. In examples, FIG. 6 depicts an embodiment of the mesh-based structure or mesh 502 also shown in FIG. 5. FIG. 7 illustrates an embodiment of a porous structured form 702, which may include micro-or macroporous architectures designed to facilitate cellular infiltration, nutrient diffusion, and vascularization. FIG. 8 illustrates an embodiment of a fibrous structure 802, which may comprise such as woven, non-woven, or electrospun fiber networks and the like without limitations. The fibrous structure 802 may provide mechanical strength and/or a biomimetic extracellular matrix-like environment for tissue integration. FIG. 9 illustrates an embodiment of a solid form, which may include, but is not limited to, a patch 902 similar to the patch 410 as previously discussed in connection with FIG. 4. This solid form or patch 902 (referred interchangeably without limitations) may provide a stable and continuous surface suitable for tissue regeneration, defect filling, or therapeutic agent delivery.

In embodiments, the body portion 202 of the therapeutic mechanism 100, upon transitioning into the structured form may form into the porous structure (also referred to as porous structured form or porous form 702) as discussed above, The porous structure 702 may include interconnected spaces within a solid structure that may facilitate nutrient exchange and/or support tissue growth by promoting cell migration and/or cell-cell signaling into pores of the body portion 202.

In embodiments, the body portion 202 of the therapeutic mechanism 100, upon transitioning into the structured form may form the fibrous structure 802 as discussed above. The fibrous structure 802 may include a plurality of networked fibres that may support tissue integration by promoting cellular alignment and/or facilitating generation of a fibrous tissue.

In embodiments, the body portion 202 of the therapeutic mechanism 100, upon transitioning into the structured form, may be formed as a hydrogel that may be responsive to environmental stimuli, such as temperature, electric current, moisture, enzymes, pH, and the like without limitations. The hydrogel may include an equilibrium water content of at least a predefined percentage value.

In embodiments, the body portion 202 of the therapeutic mechanism 100, upon transitioning into the structured form, may form as the mesh 502 as discussed above. The mesh 502 may be of a fibrous mesh type without limitations that may allow for flexibility and/or strength while providing mechanical support to soft tissue at the target site 110 during healing process or tissue generation.

In embodiments, the body portion 202 of the therapeutic mechanism 100, upon transitioning into the structured form, may be made of a composite structure that may combine one or more of a synthetic polymer and a natural polymer to provide predefined mechanical properties, biodegradation rate, or biological compatibility, among various other properties without limitations.

In embodiments, the body portion 202 of the therapeutic mechanism 100, upon transitioning into the structured form, may form a sponge-like structure with interconnected pores that may be designed to support cellular infiltration and/or nutrient exchange to facilitate in tissue generation or wound healing.

In embodiments, the body portion 202 of the therapeutic mechanism 100, upon transitioning into the structured form, may include a multi-layered structure with a plurality of layers including at least an outer layer and an inner layer. Each layer may have a varying porosity or stiffness to mimic gradient properties of natural tissues at the target site 110. The outer layer may have a higher stiffness to provide mechanical support and the inner layer may have a lower stiffness to facilitate tissue generation, in various embodiments.

In embodiments, the body portion 202 of the therapeutic mechanism 100, upon transitioning into the structured form, may include a fiber reinforcement to improve mechanical strength, flexibility, 3D-deformability, and structural support. In examples, the body portion 202 may be adapted for deployment at the target site 110 subjected to tensile stress or load-bearing forces.

In examples, the body portion 202 of the therapeutic mechanism 100, upon transitioning into the structured form, may include a coating configured to improve biocompatibility, promote cellular integration, or improve tissue adherence without limitations at the target site 110.

In embodiments, the body portion 202 of the therapeutic mechanism 100, upon transitioning into the structured form, may be formed as a porous film that may facilitate controlled delivery of nutrients or promotes tissue generation for soft tissue applications. The porous film may include a porosity structure with a pore size ranging from 1 micron to 1000 microns in various examples. The porous film may be custom selected based on type of tissue to be generated at the target site 110. The tissue may be selected from a group consisting of a skin tissue, a bone tissue, a cardiovascular tissue, a lung tissue, cartilage, an adipose tissue, a tendon, a ligament, a skeletal muscle tissue, a hepatic tissue, a pancreatic tissue, a retinal tissue, a glandular tissue, a kidney tissue, an intestinal epithelium, a meniscal tissue, peripheral axons, a smooth muscle endothelial tissue, nerve cells, fibroblasts, or microvascular epithelial cells, to accommodate varying cellular dimensions, migration requirements, and mechanical properties of the target site 110, in various examples without limitations. In example embodiments, the therapeutic mechanism 100 may promote skeletal muscle differentiation from localized satellite cells.

The porous film may further be customized based patient-specific factors such as age, gender, metabolic profile, immune response, hormonal levels, disease state, or regenerative capacity and he like without limitations, to optimize biocompatibility, cellular integration, or functional performance at the target site 110 without limitations in various examples. In examples, the porous film may incorporate microparticles or nanocarriers, selected from liposomes, polymeric microspheres, or hydrogel nanoparticles, that may be embedded within its matrix for controlled release of therapeutic agents 104 without limitations.

In examples, the body portion 202 of the therapeutic mechanism 100, upon transitioning into the structured form, may have a nano-structured surface that may improve cell adhesion or migration by providing topographical cues to migrating cells.

In some embodiments, the biocompatible ink 102 that may form the body portion 202 of the therapeutic mechanism 100 post transition may be configured to release encapsulated growth factors in a spatiotemporally controlled manner to guide tissue generation.

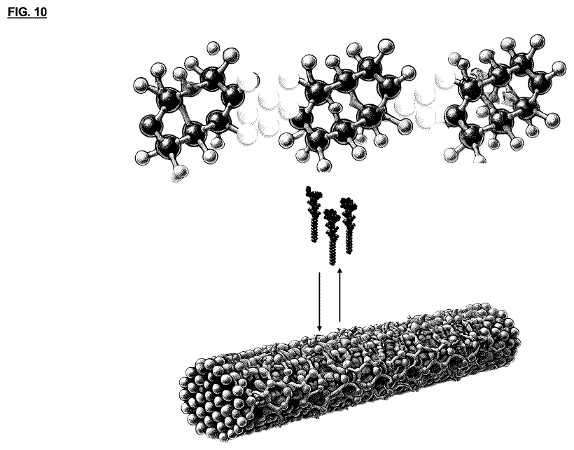

In accordance with various embodiments, the transformation of the biocompatible ink 102 into the structured form at the target site 110 may involve a series of molecular or cellular interactions that may facilitate the formation of stable and functional architectures. The transformation process may be initiated through mechanisms such as cross-linking, polymerization, ionic interactions, self-assembly, or other biochemical and biophysical cues present at the target site 110, as shown through an exemplary illustration in FIG. 10, without limitations. In example embodiments, upon administration, the biocompatible ink 102 may initially exist in the initial state, wherein various molecular components remain in a dispersed or semi-structured configuration. Following exposure to physiological conditions, such as temperature, pH, ionic strength, enzymatic activity, chemical process or presence of specific bioactive agents, the biocompatible ink 102 may undergo the transition, resulting in the formation of the structured form with predefined mechanical, chemical, or biological properties.

In embodiments, at the molecular level, the transformation process may be governed by polymeric cross-linking, such that individual polymer chains within the biocompatible ink 102 may form covalent or non-covalent bonds, which may lead to the development of a three-dimensional network, in some embodiments without limitations. In some embodiments, cross-linking may be achieved through mechanisms such as photopolymerization, enzyme-mediated cross-linking, ionic gelation, or thermal curing, and the like without limitations. In some embodiments, bioactive moieties, such as peptide amphiphiles, growth factors, or cell adhesion molecules, may facilitate cell-matrix interactions that may guide cellular attachment, proliferation, or differentiation within the structured form. The self-assembly of biomolecular components, including such as peptides, proteins, or polysaccharides, may further contribute to organization of the structured form, which may enable generation of biomimetic architectures that may support tissue regeneration and/or integration.

In embodiments, on a cellular level, the structured form resulting from the transformation of the biocompatible ink 102 may provide an environment conducive to cellular infiltration, extracellular matrix deposition, or tissue remodeling. In some embodiments, embedded or recruited cells may secrete endogenous factors that may further improve stabilization of the structured form and promote integration with surrounding host tissues. The structural or biochemical properties of the transformed bio-ink 102 may be tailored to achieve specific biological outcomes, such as osteointegration for bone regeneration, angiogenesis for vascularization, or neurogenesis for neural repair, and the like without limitations. These molecular-level interactions that may govern the transformation process may allow the biocompatible ink 102 to be configured to yield a wide range of structured forms optimized for various therapeutic applications, including those illustrated in FIGS. 6-9, without limitations.

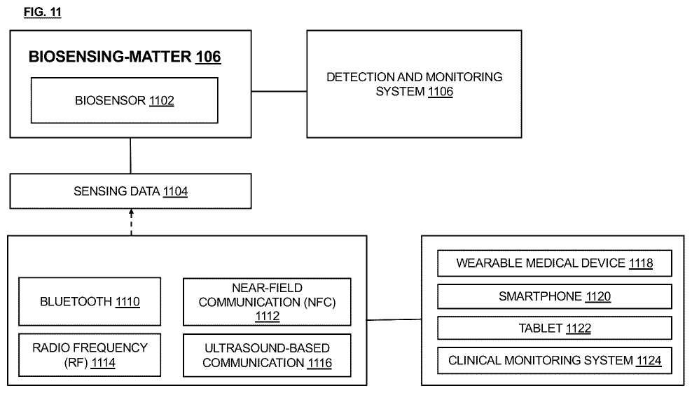

FIG. 11 illustrates the sensing matter 106 in accordance with some embodiments. The sensing matter 106 may include a biosensor 1102 selected from a group consisting of enzymatic sensors, electrochemical sensors, fluorescence-based sensors, optical sensors, or impedance-based sensors, among various other types of sensors without limitations. The biosensor 1102 may be configured to detect a biological parameter within the target recipient 112. The biological parameter may include such as pH, glucose level, lactate concentration, ATP levels, tissue thickness, implant degradation, oxygen saturation, cytokine levels, oxidative stress markers, enzymatic activity, blood flow dynamics, hemoglobin levels, electrophysiological activity, nerve conduction velocity, ion release from the therapeutic mechanism 100, ion concentrations, blood glucose levels, or inflammation markers, and the like without limitations.

In various embodiments, the biosensor 1102 may be of a variety of types without limitations such as a microelectromechanical system (MEMS)-based biosensor, nanotechnology-based biosensor, or a microfluidic biosensor among others.

In some embodiments, the biosensor 1102 may be a genetically engineered cell-based biosensor embedded within the biocompatible ink 102. The genetically engineered cell-based biosensor may be configured to express a detectable signal upon exposure to the biological parameter.

In some embodiments, the biosensor 1102 may be a thermoresponsive or pH-sensitive hydrogel-based sensor that may be configured to change its physical state in response to a variation in the biological parameter beyond a predefined threshold.

In some embodiments, the biosensor 1102 may be an electroactive-based sensor that may be configured to generate an electrical signal upon interaction with the biological parameter. The electrical signal may be measurable using an external detection and monitoring system 1106.

In some embodiments, the biosensor 1102 may be a magnetically responsive nanostructure configured to exhibit a change in magnetic properties upon interaction with the biological parameter. The change may be detectable using an external magnetic sensing device. In example embodiments, the biosensor 1102 may be compatible with MRI and other magnetic imaging stimuli.

In some embodiments, the biosensor 1102 may include living cells that may express a detectable biomarker in response to the biological parameter. The living cells may include bacterial cells, mammalian cells, stem cells, or yeast cells, among various other types of cells. The detectable biomarker may include a fluorescent protein, a luminescent marker, or a secreted metabolite indicative of the biological parameter among various other types of biomarkers without limitations.

The biosensor 1102 may be embedded within the biocompatible ink 102 to enable real-time monitoring of the biological parameter. In some embodiments, the sensing matter 106 may include an indicator that may be coupled communicatively and/or structurally with the biosensor 1102. The indicator may include such as but not limited to a fluorescent indicator, colorimetric indicator, luminescent marker, phosphorescent marker, radiographic contrast agent, thermochromic indicator, electrochromic indicator, mechanochromic indicator, quantum dot-based marker, or any other suitable type of indicator without limitations that may undergo a detectable change (referred to as change interchangeably without limitations) in the biological parameter upon interaction with the biological parameter post transition of the biocompatible ink 102 into the structured form. The change may be quantifiable using the external detection and monitoring system 1106. The external detection and monitoring system 1106 may include but is not limited to optical readers, spectrophotometers, fluorometers, Raman spectroscopes, infrared sensors, biosensor arrays, smartphone-based detection systems, electrochemical analyzers, microfluidic detection platforms, MRI or CT imaging systems, thermal imaging systems, or wireless sensor networks, among many other types of detection or monitoring devices. The sensing matter 106 may be configured to wirelessly transmit sensing data 1104 related to the biological parameter to the external detection and monitoring system 1106 via at least one of Bluetooth 1110, near-field communication (NFC) 1112, radio frequency (RF) 1114, or ultrasound-based communication 1116, among various modes of communication utilizing a variety of channels without limitations. The external detection and monitoring system 1106 may be coupled communicatively with a wearable medical device 1118, a smartphone 1120, a tablet 1122, or a clinical monitoring system 1124, and the like without limitations. The sensing data 1104 transmitted by the sensing matter 106 within the target recipient 112 may be used for remote patient monitoring or treatment adjustment, and various other applications. In example embodiments, implantable monitoring devices, such as implantable cardiac rhythm monitoring devices (e.g., Implantable Loop Recorders), implantable hemodynamic monitors, e.g. Pulmonary Artery pressure monitors, “Cardio-MEMMS”), and the like.

In some embodiments, the sensing matter 106 may be co-functionalized with the therapeutic agents 104 and the biocompatible ink 102 such that upon detecting an abnormal biological parameter, the therapeutic mechanism 100 may initiate a controlled release of the one or more therapeutic agents 104 to modulate a detected state.

In embodiments, the body portion 202, therapeutic agents 104, and the bio-sensing matter 106 of the therapeutic mechanism 100 may form as a single integrated structure in a co-fabricated architecture that may be administered through a delivery vehicle. The delivery vehicle may be configured to deposit the composition at the target site 110 inside the recipient body to form the body portion 202 in situ with a sensing and drug delivery capability. As discussed elsewhere in the document, the delivery vehicle may be the injection device 402, 3D printer 1202, and the like without limitations.

In examples, the bio-sensing matter 106 may include a population of living cells that may be encapsulated within a composition. The living cells may be responsive to a metabolic, an inflammatory, or a pathological biomarker within the target recipient's body.

In examples, the bio-sensing matter 106 may be co-fabricated with the biocompatible ink 102, such that the bio-sensing matter 106 may be homogenously distributed within the body portion 202 of the therapeutic mechanism 100 during the transition into the structured form. The bio sensing matter 106 may be configured to deliver a continuous and/or real-time monitoring signal indicative of a biological parameter at the target site 110.

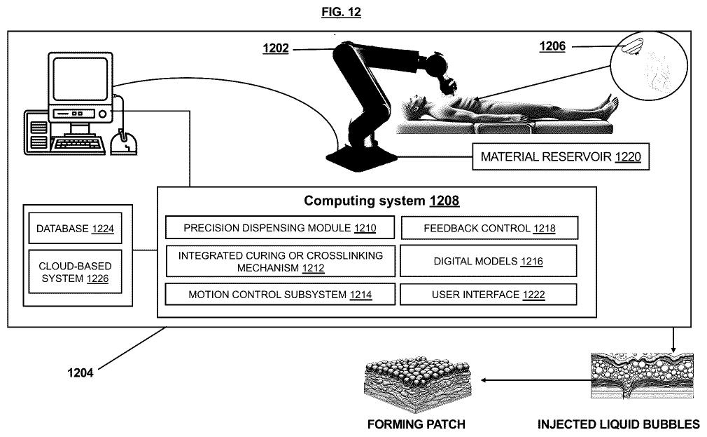

The administration involving such as delivery, deposition, or implantation of the therapeutic mechanism 100 may be achieved through various types of delivery systems or delivery vehicles, referred interchangeably without limitations. One such delivery system may be the injection device 402 as discussed in the FIGS. 4 and 5 without limitations. Another exemplary embodiment of the delivery mechanism may include a 3D printer 1202 such as shown in FIG. 12. FIG. 12 illustrates an exemplary embodiment of such a 3D printer-based delivery system 1204 configured for precision-based administration involving deposition, deployment, or implantation of the therapeutic mechanism 100 at the target site 110. The 3D printer 1202 may utilize various bioprinting techniques, such as extrusion-based printing, inkjet-based deposition, or laser-assisted printing, to achieve accurate placement and/or patterning of the therapeutic mechanism 100. The 3D printer 1202 may be designed to print various types of structured forms such as scaffolds, implants, regenerative matrices, or composite tissue constructs and others that have been discussed elsewhere in the document, without limitations. These structured forms may be deposited or printed directly at the target site 110 or onto a build platform for subsequent implantation in various embodiments.

The 3D printer 1202 may include a print head 1206 that may dispense the therapeutic mechanism 100 containing one or more of the biocompatible ink 102, sensing matter 106, therapeutic agents 104, and the like without limitations. The print head 1206 may be communicatively coupled to a computing system 1208. The computing system 1208 may include or be coupled to a precision dispensing module 1210 that may be configured to regulate flow rate, pressure, or deposition pattern of the bio-ink 102 to achieve necessary form such as the structured form. In some embodiments, the computing system 1208 may include or be coupled to an integrated curing or crosslinking mechanism 1212, such as ultraviolet (UV) light, thermal treatment, or chemical crosslinkers and the like without limitations. The integrated curing or crosslinking mechanism 1212 may facilitate in situ stabilization of the therapeutic mechanism 100 after administering to retain a desired form upon administration including such as delivery, deposition, printing, implantation and the like without limitations.

The computing system 1208 of the 3D printer 1202 may include a motion control subsystem 1214. In embodiments, the motion control subsystem 1214 of the 3D printer 1202 may be responsible for guiding the print head 1206 in multiple axes (such as X, Y, or Z directions) to enable layer-by-layer fabrication of the structured form. The movement may be controlled by one or more programmed digital models 1216. These digital models 1216 may be derived from one or more of anatomical imaging, CAD-based design files, or real-time user input. The computing system 1208 of the 3D printer 1202 may include or be coupled communicatively to a feedback control 1218 module that may utilize one or more of an imaging sensor, pressure monitor, or real-time tracking system and the like without limitations to adjust one or more deposition parameters dynamically for achieving precision or reproducibility in the printed structure without limitations.

In embodiments, the 3D printer 1202 may include a material reservoir or cartridge system 1220 and the like without limitations. The material reservoir 1220 may be configured to hold the therapeutic mechanism 100.

In embodiments, the 3D printer 1202 may be coupled communicatively to a user interface 1222 for an operator to configure printing parameters, monitor progress, make real-time adjustments or perform any other control activity without limitations. The user interface 1222 may provide visualization of an ongoing deposition process. In embodiments, the 3D printer 1202 may be integrated with a database 1224 or a cloud-based system 1226 for storing and/or retrieving print parameters in therapeutic applications.

In embodiments, the 3D printer 1202 may be adapted for in situ bioprinting, where the deposition may occur directly at various anatomical sites within a surgical or interventional setting. This may be achieved using a robotic-assisted arm, an endoscopic print head, or a handheld bioprinting device and the like without limitations.

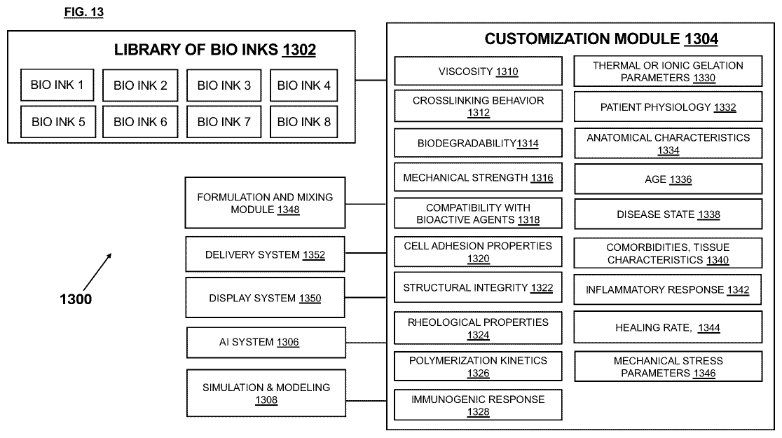

FIG. 13 illustrates an exemplary system 1300 for customizing and/or selecting the biocompatible ink 102 from a library of biocompatible-inks, for use in the therapeutic mechanism 100. The system 1300 may include a bio-ink library 1302, a customization module 1304, an artificial intelligence (AI) system 1306, a simulation and modeling system 1308, a display system 1350, and a delivery system 1352.

In various embodiments, the bio-ink library or library of bio inks 1302 referred interchangeably without limitations may include a plurality of biocompatible inks 102 such as bio ink 1, bio ink and the like as shown in FIG. 13 for an arbitrarily select number of bio inks or representation purpose without limitations. Each of the bio inks 102 may possess predefined characteristics such as viscosity 1310, crosslinking behaviour 1312, biodegradability 1314, mechanical strength 1316, compatibility with bioactive agents 1318, cell adhesion properties 1320, structural integrity 1322, rheological properties 1324, polymerization kinetics 1326, immunogenic response 1328, thermal or ionic gelation parameters 1330, and the like without limitations. The bio-ink library 1302 may store the plurality of bio-inks 102 that may be pre-formulated or may provide a mechanism for on-demand formulation using specified programmable guidelines executed through a computing system operated by an external user.

The customization module 1304 may receive one or more customization inputs, related to such as but not limited to patient physiology 1332, anatomical characteristics 1334, age 1336, disease state 1338, comorbidities, tissue characteristics 1340, inflammatory response 1342, healing rate 1344, mechanical stress parameters 1346 of the target site 110, one or more of the predefined characteristics as mentioned above such as viscosity 1310, crosslinking behaviour 1312, biodegradability 1314, mechanical strength 1316, compatibility with bioactive agents 1318, cell adhesion properties 1320, structural integrity 1322, rheological properties 1324, polymerization kinetics 1326, immunogenic response 1328, thermal or ionic gelation parameters 1330, and the like without limitations. The customization module 1304 may select or generate a bio-ink from the bio inks 102 with tailored properties to meet therapeutic and structural requirements for administration, tissue regeneration, or biofabrication applications.

In examples, the selection or generation of the biocompatible ink 102 may be based on a physiology of the target recipient 112 for receiving the therapeutic mechanism 100. In various embodiments, the physiology type may include such as blood type, age, type or severity of inflammation, resistance to cellular or immune mediated mechanisms of tissue rejection (e.g., biocompatible, bio-identical, hypoallergenic, hypoimmunogenic, histocompatible, HLA-compatible, and the like), immune response characteristics, hormone levels, endocrine, peri-endocrine, neuroendocrine, integrin, and other secretory functions, metabolic rate, resistance to calcification, thrombosis, or degradation, oxygenation levels, vascularization state of the target tissue, presence of chronic disease conditions, presence of autoimmune disorders, pH balance of the target site, enzymatic activity at the target site 110 of the target recipient 112 where the therapeutic mechanism 100 is configured to be deployed, extracellular matrix composition of the target site 110 of the target recipient 112, wound healing capability, one or more genetic markers indicative of regenerative potential within the target recipient 112, prior exposure of the target recipient 112 to immunosuppressive therapy, degree of fibrosis in the target recipient 112, hydration levels of the target site 110 within the target recipient 112, presence of at least one of a bacterial or viral infection at the target site 110 of the target recipient 112, stress, cortisol level, presence of necrotic or apoptotic cells at the target site 110, levels of circulating stem cells in the target recipient 112, prior surgical history of the target recipient 112, elasticity (e.g., viscoelasticity) or mechanical properties (and/or vascular sheer properties) of the target site 110 within the target recipient 112, and the like without limitations.

In some embodiments, the system 1300 may include an AI-based processing unit or AI system 1306 that may be configured to analyze historical data, patient-specific parameters, or real-time inputs to optimize the selection or formulation of the bio-ink 102 as per the inputs. The AI system 1306 may generate one or more recommendations based on predictive modeling, so that an optimal therapeutic outcome may be achieved with the customization.

The selected or customized bio-ink 102 may be processed through the simulation and modeling system 1308 that may evaluate an expected behavior post-administration, including structural stability, degradation profile, integration or migration with surrounding biological tissue. In embodiments, simulation results may be displayed on the display system 1350 for user review or validation before proceeding with bio-ink 102 administration including such as deployment, delivery or implantation at the target site 110 within the target recipient's body.

The customized bio-ink 102 may then be loaded into a delivery system 1352 such as the delivery systems shown in FIGS. 4, 5, and 12 without limitations. In some embodiments, the delivery system 1352 may be an injection device such as the injection device 402 illustrated in FIGS. 4 and 5. In some embodiments, the delivery system 1352 may be a 3D printer such as the 3D printer 1202 as shown in in FIG. 12. In some embodiments, the delivery system 1352 may include a combination of more than one delivery mechanisms including such as the 3D printer 1202, injection device 402, from among a variety of deposition or delivery mechanisms or systems 1352 without limitations. The delivery system 1352 may be used for precise administration of the bio ink 102 with alone or along with other components of the therapeutic mechanism 100 at the target site 110. The delivery system 1352 may facilitate controlled delivery of the therapeutic mechanism 100 based on pre-defined inputs, patterns, injection depths, or localized delivery strategies to achieve the desired structured form, such as scaffolds, implants, or tissue-engineered constructs of various forms, shapes, sizes, and the like characteristics without limitations.

In some embodiments, the system 1300 illustrated in FIG. 13 may further include a formulation and mixing module 1348 configured to receive the selected or customized bio-ink 102 and combine it with additional components of the therapeutic mechanism 100 before delivery. The formulation and mixing module 1348 may integrate such as the therapeutic agents 104, sensing matter 106, or other bioactive formulations without limitations to create a composite therapeutic composition such as the therapeutic mechanism 100 discussed throughout this document without limitations. The process of mixing may be performed under controlled conditions to maintain the stability, homogeneity, and functional integrity of the combined components. Once the complete therapeutic composition is prepared, the formulation and mixing module 1348 may transfer it to the delivery system for precise delivery at the target within the recipient 112.

In example embodiments, FIG. 13 illustrates an exemplary integrated system that may allow the selection, customization, simulation, or deployment of the bio-ink 102 for therapeutic applications, providing optimal compatibility with patient-specific biological or structural requirements, without limitations.

In accordance with various embodiments, a method for tissue generation or therapeutic monitoring may be provided. The method may utilize the therapeutic mechanism as disclosed above in conjunction with various figures. The method may include selecting the biocompatible ink 102 from the library of the biocompatible inks 102. The library may include one or more biocompatible inks 102 that may be characterized by predefined properties as discussed above in various embodiments without limitations. The selection of the biocompatible ink 102 may be based on the physiological parameters of the target recipient 112 to ensure suitability for receiving the therapeutic mechanism 100. Upon selection, the method may include administering the selected biocompatible ink 102 to the target recipient 112 through the delivery system. After administration, the selected biocompatible ink 102 may be induced to undergo a structural and/or chemical transition into the structured form at the target site 110 within the target recipient 112. The transition of the biocompatible ink 102 into the structured form may be driven by interactions occurring at the target site 110, including but not limited to biomolecular interactions, hydrophobic interactions, hydrogen bonding, ionic interactions, or exposure to polyvalent ions, and the like without limitations. The process of transformation has been discussed in conjunction with FIG. 10.

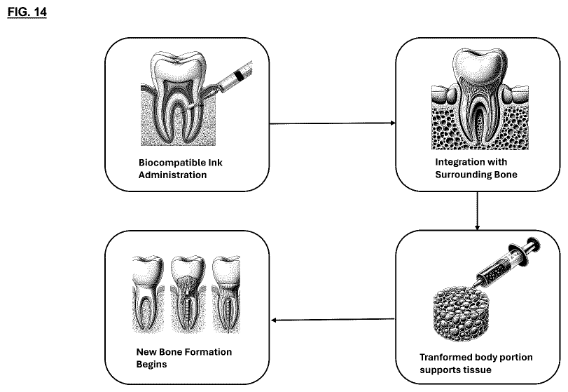

In embodiments, the body portion 202 of the therapeutic mechanism 100, upon transitioning into the structured form, may be configured for dental tissue generation and support, as shown in FIG. 14. The body portion 202 may include a scaffold that may be engineered to support one or more of alveolar bone generation, gingival tissue repair, or periodontal ligament integration, and the like without limitations. The scaffold may be infused with bioactive factors in various examples to promote osteogenesis, periodontal attachment, or microbial resistance in an oral cavity without limitations.

In some examples, the body portion 202 of the therapeutic mechanism 100 may be configured to support or generate a cardiovascular tissue, as shown in FIG, 15. In examples, the body portion 202 of the therapeutic mechanism 100, upon transitioning into the structured form, may be configured for cardiovascular tissue generation. The body portion 202, in such embodiments, may include a scaffold that may be designed to mimic extracellular matrix of a vascular wall, per-vascular structures, myocardial structure, cardiac valvular components and by promoting cell adhesion, proliferation, or differentiation to support formation of blood vessels. The body portion 202 of the therapeutic mechanism 100 may be designed to provide structural scaffolding for a heart tissue, promoting formation of a functional myocardium at the target site 110 to restore heart function post-injury, infarction, infiltration, inflammation, fibrosis of myocardium, or heart attack, without limitations in some examples. The scaffold may be engineered to provide mechanical support to the functional myocardium, thereby reducing risk of heart failure by preventing myocardial thinning, negative remodeling, fibrosis, edema, or dysfunction post-implantation, in examples.

In examples, the scaffold may be resorbed over time while being replaced by generated cardiovascular tissue, promoting long-term myocardial, vascular, and valvular generation. In examples, the body portion 202 of the therapeutic mechanism 100 may include a multi-layered structure. A first layer may promote myocardial tissue growth and repair and another layer may provide structural support to prevent mechanical and functional failure at the target site 110.

In some examples, the body portion 202 may be designed for neural tissue generation. The body portion 202 in such cases may be configured to provide structural support for growth of nerve fibers or facilitate neural tissue repair. The body portion 202 may be designed to mimic bio-mechanical properties of the neural tissue or improve nerve growth by promoting cellular alignment or tissue integration at the target site 110 of nerve injury in examples without limitations.

In examples, the body portion 202 may include a scaffold that may be configured to provide direct mechanical reinforcement to an abdominal wall at a hernia site. The scaffold may facilitate preventing hernia from recurring by providing a structural support for surrounding tissues during a healing process. In some examples, the body portion 202 may be configured to provide support to urethra for reducing urinary leakage in stress urinary incontinence.

In some examples, the body portion 202 may be configured to provide support or repositioning to a prolapsed tissue or restore anatomical alignment to prevent descent of the prolapsed tissue.

In some examples, the body portion 202 may be configured to reinforce a pelvic floor muscle, providing structural support to a weakened or damaged tissue to aid in pelvic floor repair.

In examples, various intrinsic biomechanical characteristics of the body portion 202 may be tailored to match mechanical properties of a host tissue at the target site 110 of the target recipient 112. The biomechanical and/or bio-secretory properties of the body portion 202 may be designed to vary and be customized based on individual patient factors, such as age, physiology, or gender, weight, height, body habitus, developmental stage (Pre-natal, infant, child, adolescent, adult), without limitations.

The various components described herein and/or illustrated in the figures in connection with the 3D printer 1202 and/or programmable instructions may be embodied as hardware-enabled modules and may be implemented in a hardware and/or software-based system to enhance precision, control, and monitoring of the therapeutic mechanism 100. In some embodiments, a software-controlled system may regulate the administration of the biocompatible ink 102, optimizing parameters such as injection rate, composition mixing, and transition triggers based on real-time physiological feedback from the recipient 112. In some embodiments, hardware components such as robotic-assisted injection devices, automated biofabrication platforms, or smart syringes with integrated sensors may be employed to ensure precise deposition and transformation of the biocompatible ink 102 at the target site 112.

The various components may include a plurality of overlapping or independent electronic circuits, devices, and discrete elements packaged onto a circuit board to provide data and signal processing functionality within a computer. An example might be a comparator, inverter, or flip-flop, which could include a plurality of transistors and other supporting devices and circuit elements. The modules that include electronic circuits process computer logic instructions capable of providing digital and/or analog signals for performing various functions as described herein. The various functions may further be embodied and physically saved as any of data structures, data paths, data objects, data object models, object files, database components. For example, the data objects could include a digital packet of structured data. Example data structures may include any of an array, tuple, map, union, variant, set, graph, tree, node, and an object, which may be stored and retrieved by computer memory and may be managed by processors, compilers, and other computer hardware components. The data paths may be part of a computer CPU that performs operations and calculations as instructed by the computer logic instructions. The data paths could include digital electronic circuits, multipliers, registers, and buses capable of performing data processing operations and arithmetic operations (e.g., Add, Subtract, etc.), bitwise logical operations (AND, OR, XOR, etc.), bit shift operations (e.g., arithmetic, logical, rotate, etc.), complex operations (e.g., using single clock calculations, sequential calculations, iterative calculations, etc.). The data objects may be physical locations in computer memory and may be a variable, a data structure, or a function. Some examples of the modules include relational databases (e.g., such as Oracle® relational databases), and the data objects may be a table or column, for example. Other examples include specialized objects, distributed objects, object-oriented programming objects, and semantic web objects. The data object models may be an application programming interface for creating HyperText Markup Language (HTML) and Extensible Markup Language (XML) electronic documents. The models may be any of a tree, graph, container, list, map, queue, set, stack, and variations thereof, according to some examples. The data object files may be created by compilers and assemblers and contain generated binary code and data for a source file. The database components may include any of tables, indexes, views, stored procedures, and triggers.

In an example, the embodiments herein may provide a computer program product configured to include a pre-configured set of instructions, which when performed, may result in actions as stated in conjunction with various figures herein. In an example, the pre-configured set of instructions may be stored on a tangible non-transitory computer readable medium. In an example, the tangible non-transitory computer readable medium may be configured to include the set of instructions, which when performed by a device, may cause the device to perform acts similar to the ones described here.

The embodiments herein may also include tangible and/or non-transitory computer-readable storage media for carrying or having computer-executable instructions or data structures stored thereon. Such non-transitory computer readable storage media may be any available media that may be accessed by a general purpose or special purpose computer, including the functional design of any special purpose processor as discussed above.

By way of example, and not limitation, such non-transitory computer-readable media may include RAM, ROM, EEPROM, CD-ROM or other optical disk storage, magnetic disk storage or other magnetic storage devices, or any other medium which may be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above may also be included within the scope of the computer-readable media.

Computer-executable instructions include, for example, instructions and data which cause a special purpose computer or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language and stored in a computer storage medium, such as a disk, tape, physical hard drive, or virtual hard drive such as in a storage access network. If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips may be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product may be any product that includes integrated circuit chips, ranging from toys and other low-end applications to advanced computer products having a display, a keyboard or other input device, and a central processor.

Furthermore, the embodiments herein may take the form of a computer program product accessible from a computer-usable or computer-readable medium providing program code for use by or in connection with a computer or any instruction execution system. For the purposes of this description, a computer-usable or computer readable medium may be any apparatus that may comprise, store, communicate, propagate, or transport the program for use by or in connection with the instruction execution system, apparatus, or device.

The medium may be an electronic, magnetic, optical, electromagnetic, infrared, or semiconductor system (or apparatus or device) or a propagation medium. Examples of a computer-readable medium may include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements may include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) maybe coupled to the system cither directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

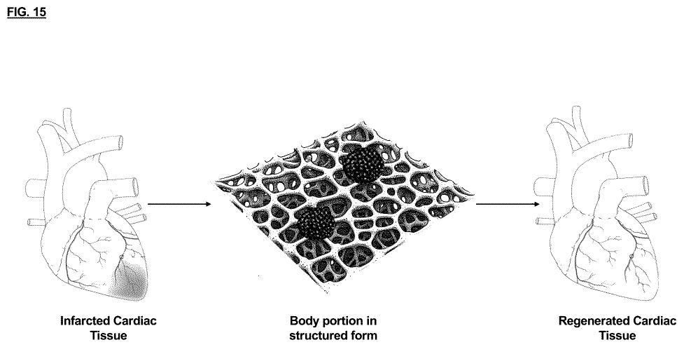

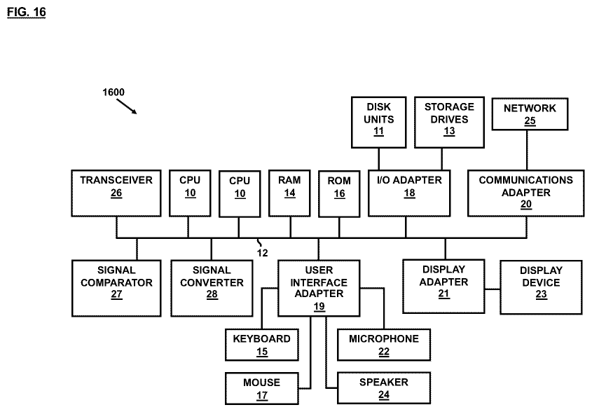

A representative hardware environment for practicing the embodiments herein is depicted in FIG. 16, with reference to FIGS. 1 through 15. This schematic drawing illustrates a hardware configuration of an information handling/computer system 1600 in accordance with the embodiments herein.

The system 1600 comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 may connect to peripheral devices, such as disk units 11 and tape drives 13, or other program storage devices that are readable by the system. The system 700 may read the inventive instructions on the program storage devices and follow these instructions to execute the methodology of the embodiments herein. The system 700 further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network, and a display adapter 21 connects the bus 12 to a display device 23 which may be embodied as an output device such as a monitor, printer, or transmitter, for example. Further, a transceiver 26, a signal comparator 27, and a signal converter 28 may be connected with the bus 12 for processing, transmission, receipt, comparison, and conversion of electric or electronic signals.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein may be practiced with modification within the spirit and scope of the present invention.

What is claimed is:

1. A therapeutic mechanism for tissue regeneration, repair, and therapeutic monitoring, comprising:
a biocompatible ink configured to transition from an initial state to a structured form in response to interaction with predefined physiological stimuli post injection into a target recipient;
one or more therapeutic agents; and a bio-sensing matter that is configured to detect at least one biological parameter of the target recipient post transition of the biocompatible ink into the structured form.

2. The therapeutic mechanism of claim 1, wherein the one or more therapeutic agents are admixed within the biocompatible ink.

3. The therapeutic mechanism of claim 1, wherein the bio-sensing matter is integrated into the biocompatible ink.

4. The therapeutic mechanism of claim 1, wherein the structured form post-transition comprises at least one of a porous form, solid structure, fibrous structure, and mesh structure allowing for at least one of cellular infiltration, vascularization, and extracellular matrix deposition.

5. The therapeutic mechanism of claim 1, wherein the therapeutic mechanism comprises an implantable device that is administered as an injectable.

6. The therapeutic mechanism of claim 1, wherein the therapeutic mechanism facilitates target recipient in vivo 3D printing of the structured form.

7. The therapeutic mechanism of claim 1, wherein the therapeutic mechanism comprises a substrate configured for in situ cell integration, such that the biocompatible ink is 3D printed on the substrate to fabricate an anatomical structure with predefined biophysical and biochemical properties.

8. The therapeutic mechanism of claim 1, wherein the biocompatible ink is selected from a library of biocompatible inks, the library of biocompatible inks comprising one or more biocompatible inks with predefined properties including one or more of viscosity, crosslinking behavior, biodegradability, mechanical strength, and compatibility with bioactive agents.

9. The therapeutic mechanism of claim 8, wherein selecting the biocompatible ink is based on a physiology of the target recipient for receiving the therapeutic mechanism.

10. The therapeutic mechanism of claim 1, wherein the biocompatible ink is at least one of structurally and chemically configured to undergo the transition into the structured form, such that the biocompatible ink defines and constitutes a substantial body portion of the therapeutic mechanism post-transition.

11. The therapeutic mechanism of claim 10, wherein the body portion of the therapeutic mechanism comprises a scaffold configured to provide structural support to at least one of a bodily tissue, bone, and cartilage, and to facilitate at least one of cellular infiltration, vascularization, extracellular matrix deposition, and osteointegration for tissue regeneration and repair.

12. The therapeutic mechanism of claim 10, wherein the body portion of the therapeutic mechanism comprises a scaffold that is designed to provide controlled, sustained release of immune-modulating agents, including one or more of cytokines, checkpoint inhibitors, monoclonal antibodies, and growth factors, configured to activate and recruit immune cells at a target site of the target recipient, promoting a localized immune response, and supporting tissue regeneration proximate to the target site where the therapeutic mechanism is administered.

13. The therapeutic mechanism of claim 10, wherein the body portion of the therapeutic mechanism comprises a biocompatible scaffold, wherein the scaffold is configured to perform at least one of:
facilitate tissue regeneration by supporting cell adhesion, proliferation, and differentiation for construction of one or more of a bone, cartilage, soft tissue, neural tissue, and a vascular structure;
provide a controlled release of one or more of therapeutic, regenerative, immunomodulatory, and anti-cancer agents for localized treatment;
modulate immune response, wherein the biocompatible scaffold promotes immune activation for at least one of cancer immunotherapy and immune suppression for one or more of transplantation tolerance and autoimmune disease management;
improve at least one of angiogenesis and vascularization by promoting formation and growth of new blood vessels for improved integration with a host tissue into the target recipient;
incorporate one or more of bioactive molecules, bioactive proteins, stem cells, growth factors, and therapeutic compounds exhibiting regenerative bioactivity to facilitate functional recovery in a tissue engineering application;
serve as a therapeutic platform for an oncology application, wherein the scaffold is configured to:
deliver one or more of chemotherapeutic and immunotherapeutic agents to a localized tumor site;
modulate a tumor microenvironment to improve immune cell infiltration;
and target cancer cells via selective apoptosis-inducing biomaterials; and provide biomechanical support by maintaining shape and mechanical stability at a target site, and wherein the biocompatible scaffold comprises a biomaterial designed to mimic mechanical properties of a supported tissue at the target site.

14. The therapeutic mechanism of claim 1, wherein the biocompatible ink comprises one or more of nanoparticles, natural polymers, synthetic polymers, ceramics, bioceramics, composites, metals, hydrogels, genetically modified materials, polymeric nanocomposites, self-assembling materials, sol-gels, hybrid organic-inorganic materials, magnetic materials, conductive materials, cell laden materials, graphene, and carbon-based materials.

15. The therapeutic mechanism of claim 1, wherein the biocompatible ink of the therapeutic mechanism, upon transitioning into the structured form, is configured for dental tissue regeneration and support, and comprises a scaffold engineered to support one or more of alveolar bone regeneration, gingival tissue repair, and periodontal ligament integration.

16. The therapeutic mechanism of claim 15, wherein the scaffold is infused with bioactive factors to promote one or more of osteogenesis, periodontal attachment, and microbial resistance in an oral cavity.

17. The therapeutic mechanism of claim 1, wherein the biocompatible ink of the therapeutic mechanism, upon transitioning into the structured form, is configured for cardiovascular tissue regeneration, and comprises a scaffold designed to mimic extracellular matrix of a vascular wall, promoting one or more of cell adhesion, proliferation, and differentiation to support formation of blood vessels.

18. The therapeutic mechanism of claim 1, wherein the one or more therapeutic agents are dispersed within the biocompatible ink, wherein the one or more therapeutic agents comprise at least one of stem cells, progenitor cells, cytokines, extracellular vesicles, bioactive peptides, growth factors, bioactive proteins, and therapeutic compounds exhibiting regenerative bioactivity and configured to promote tissue regeneration at a target site, wherein the one or more therapeutic agents are released in a controlled manner post-implantation at the target site.

19. A therapeutic mechanism for tissue regeneration and therapeutic monitoring, comprising:
configuring a biocompatible ink to transition from an injectable state to a structured form in response to interaction with predefined physiological stimuli post injection into a target recipient, the biocompatible ink admixed with:
one or more regenerative agents, and
a bio-sensing matter that is configured to detect at least one biological parameter of the target recipient post transition of the biocompatible ink into the structured form, wherein the bio-sensing matter comprises a nanostructure configured to exhibit a change in properties upon interaction with the at least one biological parameter;
administering the biocompatible ink to the target recipient; and
detecting the change in the properties using an external sensing device.

20. A method of tissue regeneration and therapeutic monitoring with a therapeutic mechanism, comprising:
selecting a biocompatible ink from a library of biocompatible inks, the library comprising inks with predefined properties including viscosity, crosslinking behavior, biodegradability, mechanical strength, and compatibility with bioactive agents, wherein selecting the biocompatible ink is based on a physiology of a target recipient for receiving the therapeutic mechanism;
administering the selected biocompatible ink to the target recipient; and
causing the selected biocompatible ink to structurally and chemically undergo a transition into a structured form, such that the biocompatible ink defines and constitutes a substantial body portion of the therapeutic mechanism post-transition, wherein causing the structural and chemical transition is a result of one or more interactions at a target site of the target recipient including: biomolecular interaction, hydrophobic interaction, hydrogen bonding, ionic interaction, or exposure to polyvalent ions.
